(19) United States
(12) Patent Application Publication
(10) Pub. No.: US 2016/0348093 A1
(43) Pub. Date: Dec. 1, 2016

(54) Mobile Solid Phase Compositions for Use in Biochemical Reactions and Analyses

(71) Applicant: 10X Genomics, Inc., Pleasanton, CA (US)

(72) Inventors: Andrew D. Price, Hayward, CA (US); Christopher Hindson, Pleasanton, CA (US); Rajiv Bharadwaj, Pleasanton, CA (US); Sukhvinder Kaur, Oakland, CA (US); Geoffrey McDermott, Livermore, CA (US)

(21) Appl. No.: 15/157,643

(22) Filed: May 18, 2016

Related U.S. Application Data
(60) Provisional application No. 62/163,238, filed on May 18, 2015.

(51) Int. Cl. C12N 15/10; C12Q 1/68

(52) U.S. Cl. CPC C12N 15/1006; C12Q 1/6806; C12Q 1/6834

(57) ABSTRACT

Compositions that include particle suspensions where such particle suspensions have characteristics for use in a variety of applications including, for example, flow restriction, reagent delivery, and use in microfluidic systems. In some compositions provided, the particle suspension include deformable particles and in particular compositions the deformable particles are beads or gel beads.

### CROSS-REFERENCE

This application claims priority to U.S. Provisional Patent Application No. 62/163,238 filed May 18, 2015 which application is herein incorporated by reference in its entirety for all purposes.

### BACKGROUND

Recent history has shown an explosion of interest in and analysis of molecular biological systems in heredity, disease pathology, epidemiology, agriculture, and a variety of other fields. Along with this has come an explosion in analytical methods and systems for analyzing highly complex biological systems, including, e.g., biochemical and cellular assay systems, high throughput genetic analysis systems, complex bioinformatics, and the like.

A variety of these analytical and/or processing systems utilize mobile, solid phase supports, e.g., particles, beads, colloids or the like, for presenting components for given analyses, and/or for interacting with reactants to purify, separate, label, or otherwise assist in the processes of the analysis. For certain applications, these mobile solid phases can benefit by meeting one or more of a number of parameters in order to improve the efficacy with which they work in those applications. The present disclosure describes methods, compositions and systems that meet these and other requirements.

### SUMMARY

Described herein are improved compositions that comprise particle suspensions where such particle suspensions have novel and useful characteristics for use in a variety of applications including, for example, reagent delivery, and use in microfluidic systems.

In an aspect, the disclosure provides a composition comprising a suspension of particles. The suspension of particles can be characterized by having one or more of: (i) a dispersity of particles where at least 95% of the particles in the suspension have a particle size that is within 10% of a mean particle size for the suspension; (ii) a plurality of particles having an elastic modulus of between about 5 kPa and 100 kPa; (iii) a solution viscosity of between about 0.1 cP and about 100 cP; and (iv) particles in the suspension having a pore size of from about 1 nm to about 20 nm.

In general, in one aspect a composition is provided including a suspension of deformable particles, the suspension being characterized by one or more of:

(i) a dispersity of particles where at least 95% of the deformable particles in the suspension have a particle size that is within 10% of a mean particle size for the suspension;

(ii) a plurality of deformable particles having an elastic modulus of between about 5 kPa and 100 kPa;

(iii) a solution viscosity of between about 0.1 cP and about 100 cP; and

(iv) deformable particles having a pore size of from about 1 nm to about 20 nm.

In one embodiment the deformable particles are passable through a microfluidic physical feature narrower than the deformable particles.

In another embodiment the microfluidic physical feature is: a filter, an obstacle, a passage, a channel, a space or any combinations thereof.

In one embodiment the deformable particle is a bead. In a related embodiment the bead is a gel bead.

In another embodiment the deformable particle has a diameter selected from the group consisting of about 1 μm to about 1 mm, about 10 μm to about 100 μm, about 20 μm to about 100 μm, about 30 μm to about 80 μm and about 40 μm to about 60 μm in diameter. In one embodiment the deformable particle has a diameter of about 10 μm to about 100 μm. In another embodiment the deformable particle has a diameter of about 30 μm to about 100 μm.

In a further aspect the suspension of deformable particles is characterized by a shear modulus between about 5 kPa to about 100 kPa.

In another aspect a method of removing contaminants from the suspension of deformable particles is provided, including:

i) passing the deformable particles through a mesh filter having a pore size smaller than the diameter of the deformable particles; and

ii) collecting the deformable particles.

In a particular embodiment the mesh filter comprises a pore size of about 10 μm to about 50 μm. In a related embodiment the deformable particle has a diameter of about 10 μm to about 100 μm. In a different embodiment the deformable particle has a diameter of about 30 μm to about 100 μm. In a specific embodiment the pore size is about 30 μm. In a more specific embodiment the pore size is about 41 μm. In one embodiment the deformable particles are gel beads.

In general, in another aspect a method of storing an oligonucleotide labeled deformable gel bead composition is provided including:

i) providing a composition of deformable gel beads linked to an oligonucleotide; and

ii) storing the composition at about pH 7.4 for at least 12 weeks, wherein release of linked oligonucleotides is at most 0.025%.

In one embodiment the deformable gel bead has a diameter of about 10 μm to about 100 μm. In a specific embodiment the deformable gel bead has a diameter of about 30 μm to about 100 μm.

In general, in another aspect a composition is provided including a suspension of deformable particles characterized by:

i) the suspension having a solution including a ligation buffer, a ligase enzyme, oligonucleotides and an absence of any reducing agent, wherein the solution supports ligation of oligonucleotides to the deformable particles even in the absence of the reducing agent;

ii) the deformable particles having an elastic modulus of between about 5 kPa and 100 kPa; and

iii) the deformable particles being resistant to aggregation, wherein the deformable particles would otherwise be prone to aggregation in the presence of a reducing agent.

In one embodiment the suspension is further characterized by one or more of:

i) a dispersity of particles where at least 95% of the particles in the suspension have a particle size that is within 10% of a mean particle size for the suspension;

ii) a solution viscosity of between about 0.1 cP and about 100 cP; and

iii) particles having a pore size of from about 1 nm to about 20 nm.

In general, in a further aspect a method of filtering using the suspension of deformable particles described above is provided including:

i) using the suspension of deformable particles as a flow restrictor; and

ii) passing a solution to be filtered through the suspension.

In one embodiment the deformable particles have a pore size of from about 2 nm to about 6 nm. In a particular embodiment the deformable particles have a pore size of from about 5 nm. In a specific embodiment the deformable particles provide a size cut off of less than 4.4 nm.

In general in a further aspect a composition is providing including a suspension of particles, the suspension being characterized by having one or more of:

(i) a dispersity of particles where at least 95% of the particles in the suspension have a particle size that is within 10% of a mean particle size for the suspension;

(ii) a plurality of particles having an elastic modulus of between about 5 kPa and 100 kPa;

(iii) a solution viscosity of between about 0.1 cP and about 100 cP; and

(iv) particles having a pore size of from about 1 nm to about 20 nm.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1A-1C is a series of photographic images of gel bead particles flowing in a microfluidic system.

FIG. 2 is a plot showing the deformation effect of increasing osmotic pressure on gel bead particles size.

FIG. 3 is a plot showing the effect of pH on contamination rate over time.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The present disclosure generally provides compositions and methods for use in biochemical reactions including as part of a broader biochemical and/or biological analysis process. The compositions and methods described herein utilize mobile solid phase compositions to efficiently deliver reagents to a reaction of interest, sometimes, in a microfluidic context. The methods and compositions described herein can have use in generating highly parallelized reaction systems for analysis of highly multiplexed samples, including, for example, in nucleic acid analysis and sequencing applications.

### I. General Characteristics

Described herein are mobile solid phase compositions for use in processing and/or analytical reactions for biological, biochemical, and/or chemical processing and/or analyses. In some cases, provided are mobile solid phase systems that are used to carry and present and/or deliver reagents within microfluidic systems. In some cases, the compositions and systems described herein are configured to meet one or more of a number of different parameters that benefit the use of such compositions as reagent delivery vehicles or other uses in microfluidic systems.

In general, the characteristics of the compositions can relate to aspects such as flow characteristics within microfluidic systems, mechanical robustness of the compositions relevant to use in microfluidic systems and for the handling of iterative analysis processes, reagent loading, availability and releasability of reagents within the compositions, chemical make-up and stability, compatibility with reaction conditions, and tunability of the compositions.

The compositions described herein may generally meet one, several or most of the parameters described herein, depending upon the desired application. Generally, the particle compositions may be produced from a variety of different materials in order to meet the desired parameters. For example, in some cases, polymeric materials are employed as a matrix that forms a particle composition herein. In some cases, polymer meshes, entangled polymers, and the like are used as they provide high surface areas for attachment or association with reagent compositions. In certain aspects, hydrogel polymers are employed as the underlying matrix for the particle compositions. Polyacrylamide polymers are useful as the polymer materials used in the bead compositions, including for example, linear polyacrylamides, cross linked linear polyacrylamides, and the like. Examples of such polyacrylamides include, for example, linear polyacrylamides incorporating N,N′-Bis(acryloyl)cystamine (BAC) monomers to provide crosslinking groups. In other cases, inorganic particle materials may be used, such as silica based particles.

Particles of the compositions described herein, for example, gel beads, can be used as described herein in a range of sizes. It is envisioned that gel beads can be sized between 1 μm to 1 mm, 10 μm to 100 μm, 20 μm to 100 μm, 30 μm to 80 μm or 40 μm to 60 μm in diameter. Exemplary diameter sizes of beads include but are not limited to: 30 μm, 40 μm, 50 μm, 55 μm, 57 μm, 60 μm, 64 μm, 70 μm, 72 μm, 75 μm, 100 μm, 125 μm, 150 μm, 200 μm, 250 μm, 500 μm, 750 μm and 1 mm.

### II. Flow Characteristics

Use of mobile solid phase components within microfluidic systems relies upon the flow characteristics of those solid phase components within microfluidic channels and channel networks. In some cases, reliability of flow, flow rates, spacing and packability of particles, can all impact how these solid components move through microfluidic channels. In general, where particles are being passed through microfluidic systems, the microfluidic channels may be adapted to operate within the existing parameters of the particles employed, rather than configuring particles to better operate within the microfluidic context. The present disclosure, on the other hand, is directed to the parameters of the particle compositions, that can be provided to yield better performance in microfluidic contexts

A. Dispersity

In certain applications, the predictability of how these materials move through microfluidic channels and channel networks, e.g., the rate at which these beads flow through that system, the regularity with which they reach their destination, and the ability to avoid failure events, such as channel clogging and other failures, can dramatically impact the performance of the overall system. For example, where one wishes to allocate individual beads or particles to different droplets being formed in a microfluidic system (see, e.g., U.S. Patent Publication No. 2015/0005199, the full disclosure of which is herein incorporated by reference), the ability to precisely deliver a desired number of beads into a partition, e.g., one and only one, is dictated by the flow characteristics of those beads through the microfluidic system.

A variety of specific characteristics can impact the flow characteristics of the particles described herein. For example, the heterogeneity of the particle size in the particle composition, also referred to as dispersity, can impact flow characteristics of the particle suspension through microfluidic channels, as different size particles can have differing flow characteristics, and give rise to differing potential failure modes. In accordance with the compositions described herein, the particle suspensions can have a substantially monodisperse population size, which, as used herein, means that at least 80% of the particles in the suspension will be +/−10% of the mean particle size for the population. In some cases, at least 90% of the particles in the suspension will be +/−10% of the mean particle size for the population. In still other cases, at least 95% of the particles in the suspension will be +/−10% of the mean particle size for the population, and in still other cases, at least 98% or even 99% of the particles in the suspension will be +/−10% of the mean particle size for the population. As used herein, particle size can generally be measured as the average diameter of the particle.

Particle dispersity measurement may generally be achieved by any of a variety of known methods. For example, for high throughput measurements, e.g., measurements of 1000 or more particles, automated microscopy (e.g., using a Morphologi G3 system), dynamic imaging analysis (e.g., using a flow monitoring camera system), and light scattering (e.g., using a Mastersizer 3000 system), may be used. In some cases, the level of dispersity may be measured in terms of the standard deviation from the mean, stated as % CV.

In some cases, achieving the desired particle size can be accomplished through one or more of tightly controlled preparation techniques, as well as post preparation sorting and sizing techniques, e.g., filtration or sieving techniques. In certain cases, however, the nature of the particles may prevent use of simple size exclusion based separation techniques for the particle populations. For example, in the case of highly elastic or deformable particles, e.g., as described in greater detail herein, sieving or filtration techniques may be ineffective, as they can be more susceptible to clogging and fouling. Additionally, with elastic particles, the ability to deform and pass through smaller openings results in a much broader size distribution. Accordingly, in some cases, the particle populations may be subjected to alternate methods of size separation/selection. For example, in a first case, a population of particles may be subjected to a flotation filtration approach to particle size selection. In such cases, a solution or suspension of the particles can be provided in a floatation chamber with an upward flow rate applied through the chamber. The flow rate can be selected such that gravitational settling of heavier larger particles or aggregates overcomes the upward flow and these particles sink or at least fail to reach an elution port at an upper portion of the chamber, where properly sized particles can be removed.

In an alternative, but related approach, size selection may be carried out using vector chromatography methods and systems. In such systems, longer channels or conduits can be provided through which the suspension of particles is passed. Due to their size and ease of their diffusion across the flowing stream, smaller particles can spend greater amounts of time in the center of the flow channel at which the flow rate is greatest. Larger particles can tend to diffuse more slowly, and as a result, can spend greater amount of time in more slowly moving portions of the flow. Provided a long enough channel, smaller particles can tend to emerge from the system first.

B. Elasticity

An additional property of the compositions of the disclosure relating to their flow characteristics through microfluidic systems is their elasticity or deformability. In some cases, for applications in which particle compositions approach or even exceed one or more of the cross sectional dimensions of the microfluidic channels (or portions thereof), the ability for those compositions to pass substantially unimpeded directly impacts the flowing of those suspensions through the fluid network. Accordingly, the particle compositions described herein can be relatively elastic and/or deformable. In some cases, the particle compositions described herein will have an elastic modulus of from about 5 to about 100 kPa. In some cases, the compositions will include an elastic modulus of between about 5 and about 50 kPa, or from 50 to about 100 kPa. In still other cases, the particle compositions will have an elastic modulus of from about 5 to about 10 kPa, from about 10 to about 20 kPa, from about 20 to about 30 kPa, from about 30 to about 40 kPa, from about 40 to about 50 kPa, from about 50 to about 60 kPa, from about 60 to about 70 kPa, from about 70 to about 80 kPa, from about 80 to about 90 kPa, from about 90 to about 100 kPa. The elastic modulus of the particle compositions may be characterized using any of a variety of known methods, including, e.g., osmotic pressure compression methods, micromechanical deformation techniques, and centrifugal compression methods

Controlling the elastic modulus of the particle compositions can be accomplished through control of the manufacturing process and or composition of the solid phase or particle component of the composition. For example, for polymeric particles, elastic modulus may be adjusted by increasing or decreasing the level of packing of the polymer within the particle, e.g. by controlling secondary structure of the polymer matrix, controlling intra-molecular and intermolecular electrostatic interactions or by controlling the level of crosslinking or other structures that provide increased rigidity to the particle. For example, in the case of linear polyacrylamide polymer particles, one may increase or decrease the level of crosslinking in the particle, e.g., through increasing or decreasing the level of crosslinker components in the polymer, e.g., bis-acrylamide copolymers and cross-linking initiating reactants, e.g., TEMED.

C. Aggregation/Adhesion

In addition to the foregoing, another important parameter of the particle compositions is their ability to avoid adhesion to other surfaces within the system, including, e.g., other particles in the compositions as well as surfaces of microfluidic channels, wells, tubes, or other containers. In terms of adhesion to other particles, the compositions described herein can be non-aggregating, meaning that fewer than 10% of the particles in the composition will be in the form of an aggregate of two or more particles when suspended in aqueous solutions or when moving through a microfluidic system in an aqueous environment, e.g., including without limitation, within an aqueous droplet in a non-aqueous carrier fluid. In some cases, fewer than 5%, fewer than 4%, fewer than 3%, fewer than 2% or even fewer than 1% of the particles will be present as aggregated particles.

In some cases, one may wish to measure bead-bead interaction forces using more specific tools. For example, in some cases, bead-bead attraction may be measured using extensional flow systems applied to aggregated particles where well controlled flow regulators may be used o direct well controlled forces to determine the interactive forces.

Controlling inter-particle interactions, e.g., adhesion and interaction may be carried out using a number of different approaches, including, for example, controlling surface charge, hydrophobicity/hydrophilicity, presence of reactive functional groups on the surface.

D. Interaction with Other Surfaces

The particle compositions may also have reduced propensity to adhere to surfaces of a system in which they are disposed, e.g., microfluidic or other conduits, reaction vessels, wells, tubes or the like. As with inter-particle aggregation, as discussed above, in certain cases, important particle properties relate to their inertness to different surfaces, e.g., in order to avoid surface adhesion. The ability to prevent fouling of surfaces, clogging of channels and the like can be of concern in microfluidic systems. Likewise, in reaction systems in which reactants and/or products are present at relatively low levels, non-specific adsorption of reactants or products to vessel surfaces can skew analysis results of those reactions, e.g., by hiding reactants or products. As such, configuring the particles to be inert to, or in some cases, actively repelling to the surfaces of the reaction vessels may be desirable. This may be accomplished by a number of means, including, e.g., selecting particles having a net charge that is opposite to that of the reaction vessel surface. In certain cases, the particle compositions are provided so as to be generally hydrophilic and generally uncharged. In some cases, this can be accomplished by creating the particle compositions from uncharged and hydrophilic polymers. Non-limiting examples of polymers include, e.g., polyethylene glycol polymers (PEGs), polyacrylamide polymers, such as linear polyacrylamides, cellulose polymers, dextrans, and the like.

E. Solution Viscosity

In addition to properties of the individual particles within a population of particles, the particle compositions described herein can have bulk properties that meet certain useful parameters. In some cases, as will be appreciated, separate from the flow characteristics of individual particles flowing through a microfluidic system, the bulk viscosity of a particle suspension can also be an important flow characteristic of the particle suspension compositions described herein. In some cases, viscosity of the particle containing composition may be controlled within desired parameters in order to achieve any of a variety of different objectives. For example, in some cases, it can be desirable to have the particle containing composition have a bulk viscosity that is similar to that of other fluids being combined within microfluidic systems, in order to promote consistent flow rates among the different fluids, as well as allow for more consistent fluid mixing. Conversely, in some cases, the bulk viscosity may be controlled to provide substantially different fluidic characteristics of the particle containing composition, in order to prevent rapid mixing, in order to provide for differential flow rates, or the like.

In general, the rheology or viscosity of the particle containing compositions can be between about 0.5 centipoise (cP) and about 5000 cP. In some cases, the solution viscosity can be from 0.5 to 100, from 0.5 to 50 cP, from 1 to 50 cP, and in some cases from 1 to 10 cp. While in other cases, the solution viscosity may be higher, e.g., from 100 to 1000 cP, from 100 to 500 cP, and the like. In certain cases, the viscosity can aim to be similar to that of other fluids processed within common microfluidic systems. In general, such fluids include aqueous fluids, reagents, and the like as well as partitioning fluids, e.g., fluorinated oils and surfactants. In general, these fluids can have a bulk viscosity in the range of from about 0.5 cP to about 20 cP. As such, it may be desirable in some cases to provide the particle containing compositions when deposited in the microfluidic systems, having a viscosity of between about 0.5 cP and about 20 cP, between about 1.0 and about 10.0 cP, or even between about 1.0 cP and 5.0 cP.

In general, one may adjust the rheology of the particle containing compositions by adjusting one or more of a number of different parameters, including adjusting particle elasticity, particle-particle interactivity, particle size distribution, particle concentration, temperature, or through use of viscosity modifying additives.

### III. Mechanical Robustness and Handle-Ability:

In addition to understanding how mobile particle phases move through microfluidic systems, another important parameter for the particle systems described herein relates to their robustness under typical use. In some cases, whether it is in the context of flowing these particle suspensions through microfluidic systems, or through routine handling, e.g., pipetting, centrifuging re-suspension and agitation, freezing and thawing, the particle compositions described herein can remain substantially intact unless and until an appropriate stimulus is applied to disrupt the particles where desired. In general, robustness of the particle compositions is generally measured by virtue of the level of resulting dispersity following mechanical handling processes. For example, the particle compositions can retain the dispersity metrics described above, even following one or more pipetting steps, microfluidic injection/movement steps, centrifuge steps, vortexing steps or other mechanical handling steps.

Additionally, the particle compositions can possess such characteristics as to facilitate handling in general, e.g., appropriate density to allow proper flow characteristics while also allowing for centrifugation based separation techniques. In general, particle compositions may have a density of between about 1.001 and 1.2 for hydrated particles in a substantially aqueous medium, e.g., having a density of from 1.00 to 1.10.

### IV. Reagent Loading and Availability:

In some cases, the particle compositions described herein may be used as reagent delivery vehicles to precisely deliver a reagent payload to a desired location within a microfluidic system. As will be appreciated, this implicates important particle parameters relating to the ability to load reagents into these particles and ability to access and/or release those reagents once delivered. For a number of applications, the particles used herein comprise porous structures that facilitate both reagent loading and access to reagents within assay systems. Such porous particles may include any of a variety of porous structures, including, e.g., porous solid or semisolid structures, macromolecular matrix-like structures (e.g., entangled polymer matrices, crosslinked polymer mesh structures, and the like)

A. Mesh Size

As will be appreciated, for porous particles, the ability of materials to move into and out of the particle may be governed, in part, by the mesh size and relative porosity of the particle. In some cases, larger compounds or materials will more readily diffuse into and out of larger pore particles than for smaller pore particles, allowing for more rapid dispersion from or penetration into the particles. In some cases, it can be desirable that relatively large macromolecules be able to efficiently pass into and out of the particles.

By way of example, in some cases, particle compositions may be provided with reagents coupled to their interior matrices. In cases where a chemical stimulus is used to cause the release of the reagents form the particles, it can be desirable to allow the efficient diffusion of the stimulus into and the reactant out of the particle.

By contrast to the above, in other aspects, it may be desirable to provide the particles with a mesh size that allows smaller molecules to efficiently move into and out of the particle, while impeding the diffusion of larger molecules, e.g., macromolecules like oligonucleotides, proteins, etc.

As such, it can be desirable that the particles have a mesh or pore size of from 1 to 20 nm, in some cases 1-10 nm, in some cases 1-5 nm, in some cases, 1-4 nm, in some cases 1-3 nm, in some cases 1-2 nm. In other cases, the pore size may be from 5-20nm, from 5-10 nm, or even from 7-10 nm. In cases where it is desirable to prevent smaller molecules from diffusing into and/or out of the pores, pore sizes of smaller than 1 nm may be desirable, e.g., between 0.01 and 1 nm. Adjustment of pore sizes may be achieved by a number of methods, including, for example, by increasing the concentration of polymer present in a polymer matrix, by adjusting the level of crosslinking in a polymer matrix, and/or by changing the osmotic forces on a polymeric polymer, to cause contraction of the polymer matrix to reduce pore sizes.

B. Surface Area

As noted previously, for many applications, the particle compositions described herein can be useful as reagent delivery systems. While in some cases, the reagent delivery aspects may be provided by impregnating the particles with the reagents to be delivered, where such reagents can be retained by physical barriers, or by virtue of solvent incompatibility with their environments. However, in some cases, the reagents will be chemically coupled to the matrix that makes up the particles, e.g., through covalent or non-covalent molecular interactions. As such, in some cases, the particle compositions will sufficiently large surface areas or sufficient numbers of coupling sites, to achieve the desired loading capability on a per particle basis.

D. Molecular Crowding/Confined Reactant Space

Another advantage of using porous particles provides for the ability to enhance local concentrations within the confines of the particle relative to the surrounding carrier fluids. In some cases, it will be desirable to provide porous particles that force close interaction between reactants contained therein, despite the relative dilution of those reactants in the overall medium.

E. Responsiveness to Stimuli, Dissolvability

As described previously, in some cases, the particles may be configured to release their reagent payload upon application of a particular stimulus. In some cases, in addition to or as an alternative to reagent release, the particles may be configured to be dissolvable or degradable upon application of a stimulus, in order to facilitate reagent release, e.g., whether by release from entrainment, or by chemical dissociation from the particle matrix.

In some cases, the particle compositions described herein will be configured to release their reagent payload, and/or dissolve substantially within a desired timeframe. In some cases, the particle compositions will release at least 90% of their reagents within a desired timeframe from having been exposed to an appropriate stimulus, e.g., a chemical stimulus such as a reducing agent, optionally including elevated temperature, e.g., 95° C. In some cases, at least 95% of the reagent payload will be released, at least 98% or even at least 99% of the payload will be released. In some cases, the desired timeframe from exposure to a stimulus to release will be less than 20 minutes, less than 10 minutes, less than 5 minutes, less than 3 minutes, less than 2 minutes, less than 1 minute. In some cases, the desired timeframe will be as described above, but greater than 1 second, greater than 10 second, greater than 20 seconds, greater than 30 seconds, greater than 40 seconds, greater than 50 seconds.

Attachment of oligonucleotides to gel beads can be through a number of approaches, including but not limited to: disulfide linkage, ester linkage, silyl ether linker (see for example, US Patent Application Publication No. US 20130203675), biological linkers, UV cleavable linkers, etc.

A stimulus can be used to control release of oligonucleotides from gel beads. One approach for controlling release is to alter pH conditions. Another approach is through the action of one or more reducing agent (e.g., for releasing disulfide linkages).

### V. Chemical Make Up.

In a number of applications, an important characteristic of the particle compositions relates to how they interact with their chemical environment. In some cases, these compositions may be exposed to a wide variety of chemical conditions, such as extremes of pH, ionic strength, polar reagents, and the like. In some cases, it will be desirable for these compositions to remain relatively static, as to their characteristics, while in other cases, it will be desired that the changed environment stimulates a change in the characteristics of the particles, e.g., to release reagents that are bound thereto.

In some cases, a number of applications of the compositions described herein subject the particles to widely varying environmental and/or mechanical conditions, and the compatibility of these particles with those environments is an important characteristic.

A. Compatibility with Emulsion Chemistry

Because the particle compositions described herein can be useful as reagent delivery systems for reactions of interest, it follows that these particles can generally be compatible with the relevant reaction conditions. For many applications, such compatibility can include particle compositions that do not interact with reagents in a way that negatively impacts the underlying reaction. In some case, compatibility can be achieved via the use of particle compositions that do not have excessive charge, hydrophobicity, hydrophilicity or polarity, other than as tolerated by the given reaction system. In some cases, the particle compositions may include substantially non-ionic matrices when used in a substantially neutral pH environment, e.g., between about pH 6 and about pH 8. In some cases, the zeta potential of the particles within the particle composition will be +/−0-5 mV, when at the above described pH range.

In some cases, however, particle compositions will be subjected to relatively obscure environmental conditions. In some cases, the particle compositions will be used to co-partition the reagents to be delivered into aqueous droplets in a non-aqueous carrier fluid. In such cases, fluorinated oils can be used as the carrier fluid in which the droplets are formed. In such cases, the aqueous phase or droplets can often contain relatively high concentrations of polar compounds or surfactants, that operate to stabilize the droplets in the non-aqueous carrier fluids, i.e., to reduce their susceptibility to coalescence with other droplets. As will be appreciated these large polar surfactant compounds can have significant negative impacts on the surfaces of materials, such as particles, by fouling them, rendering them inaccessible to other materials, etc.

The particle compositions (e.g., gel beads) may be partitioned into droplets such that at least one partition comprises a particle. This may be true for about 1%, 5%, 10%, 20%>, 30%>, 40%, 50%, 60%, 70%, 80%, 90%, or more of the partitions. This may be true for at least about 1%, 5%, 10%, 20%, 30%, 40%, 50%, 60%, 70%, 80%, 90%, or more of the partitions. This may be true for less than about 1%, 5%, 10%, 20%, 30%, 40%, 50%, 60%, 70%, 80%, or 90% of the partitions.

Terminal dilution of particles, such as beads or gel beads, may achieve the loading of one particle per one droplet or any desired number of particles per droplet. In some cases, a Poisson distribution is used to direct or predict the final concentration of particles or beads per droplet.

An advantage of the particle compositions described herein relates to the deformability of the particles that favor achieving a Poisson distribution. Specifically, the physical feature of deformability of the particles permits better control over the formation of droplets bearing one or more particle. Another advantage relating to the deformability of particles in the particle compositions described herein is the improved ability to co-partition within droplets. It is envisioned that this advantage supports improved co-partitioning of particles with cells, cell components or other particles, either cellular or molecular in nature. In one embodiment, improved co-partitioning of a single particle and a single cell is provided.

B. Ionic Strength and pH Tolerance

As with respect to the polar surfactants discussed above, the particle compositions may, in some cases be subjected to environments that have widely varying ionic strengths and pH. In such cases, it may be desirable for the particle compositions to not only retain their underlying structure, e.g., non-dissociating and non-dissolving, but also substantially maintain their volume. Such tolerance may be imparted by a number of methods, including adjusting the level of crosslinking, as noted above, adjusting the level of charged monomers in the polymers, and the like.

C. Responsiveness to Stimuli

By contrast to the above, in some cases, the particle compositions described herein will be used to release a reagent payload upon application of a particular chemical or physical stimulus, and the ability of this to occur efficiently and evenly can be an important characteristic. Many of the parameters described above contribute to the responsiveness of the particle compositions, including, e.g., mesh size, surface area, monodispersity. By way of example, the responsive characteristics may include the ability to release reagents by cleavage of a chemical connection, the enlargement of a pore network to dis-entrain larger molecules, or the ability to dissolve. In accordance with these types of particle compositions, and as noted above in some cases, the particle compositions can achieve complete or substantially complete release of reagents from the particles within 10 minutes of application of the appropriate stimulus, e.g., contact with a chemical stimulus or exposure to a thermal or mechanical stimulus. By substantially complete release is meant that the particles can release at least about 50% of its reagent capacity within the timeframe described, in some cases, at least 60%, in some cases at least 70%, in some cases at least 80%, while in still other cases at least 90% or even at least 95% or 99% of its reagent carrying capacity. In some cases, the substantial release will occur in from 1 second to 10 minutes, while in some cases, it may take fewer than 8 minutes, fewer than 6 minutes, fewer than 5 minutes, fewer than 4 minutes, fewer than 3 minutes, or less. Measuring reagent release can generally be accomplished by detecting the free or unbound reagent dispersed in a fluid volume using any of a number of methods useful for measuring the concentration of the given reagent. Such methods may include steps for the separation of any particle components in order to separate free from bound reagent. This may be compared against a known or theoretical amount of reagent capacity for the particles, or compared to a long term release, e.g., of 1, 2, 3, 4, 5, 12 or more hours, as a proxy for total reagent capacity, e.g., compare a 10 minute release to a 12 hour release to provide the ration of reagent released to reagent capacity.

### VI. Tunability of Composition

As will be appreciated from the foregoing, the particle compositions can often include one, two, three, four or more of the above described characteristics, which can be selected and adjusted to accomplish a desired reaction goal. These compositions generally share the benefit of being highly tunable in all of the above describe characteristics.

### VII. Deformability of Particles

Deformability of particles, for example, gel beads, is advantageous in a microfluidic system. For example, deformable particles can pass features such as constrictions, obstacles, filters or other physical features of a microfluidic system because of their deformability or elastic nature. It is even possible for deformable particles to pass regions, spaces, filters, obstacles or other physical features having passages narrower than the deformable particle itself.

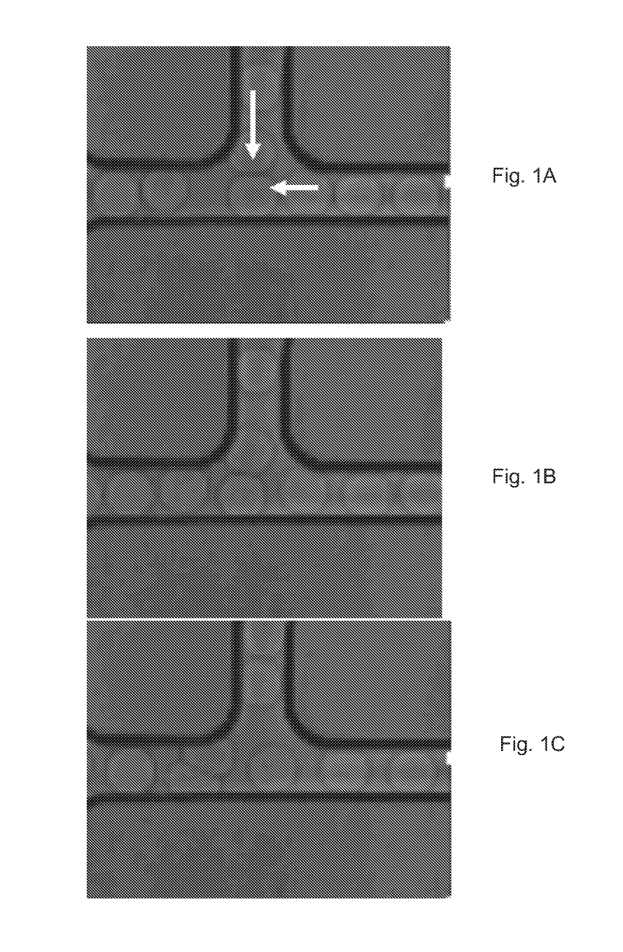

FIG. 1A-C shows deformable particles, gel beads, in a time lapse series of photomicrographs, demonstrating the gel bead's deformability. The white arrows in FIG. 1A show the direction of gel bead flow and two gel beads in a t-intersection of a microfluidic system. In FIG. 1B, the two beads move further together. In FIG. 1C the two beads clearly deform as they proceed past the t-intersection.

### VIII. Compressive Elastic Modulus

One measure of particle deformability is compressive elastic modulus (K). It is envisioned that useful particle (e.g., gel bead) deformability can be obtained in a range of K values. For example, from 0.1 kPa to 200 kPa. More specifically, in a range of 1 kPa to 100 kPa. Other useful ranges can include 10 kPa to 100 kPa, 25 kPa to 100 kPa, 25 kPa to 75 kPa and 30kPa to 65 kPa.

Particle compressive elastic modulus (K) was measured for different sized gel beads using a dextran-based osmotic pressure approach.

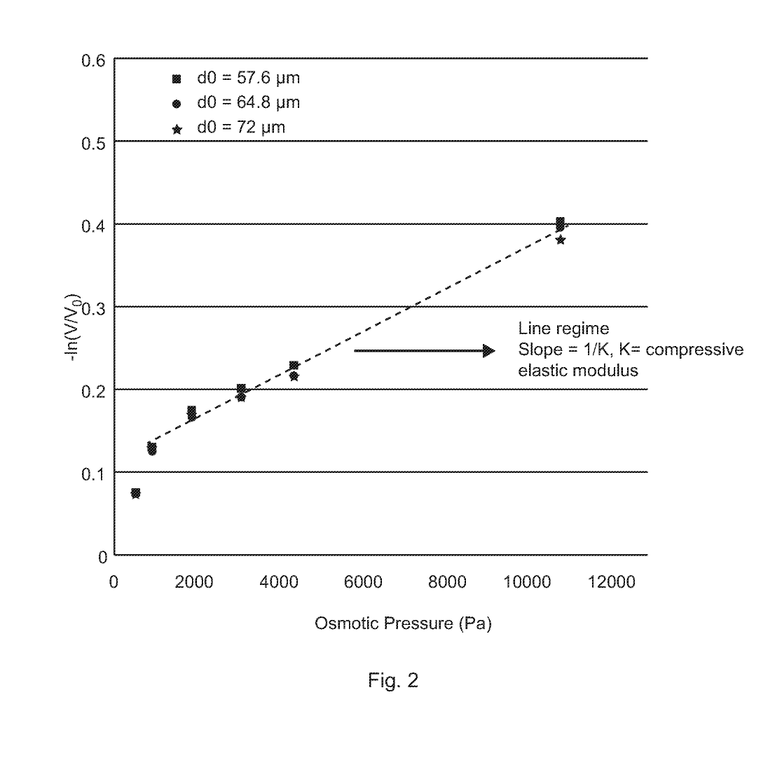

Experimental: Gel beads of three sizes, 57.6 μm, 64.8 μm and 72.1 μm were tested for their respective compressive elastic modulus (K). Gel beads were exposed to varying concentrations of dextran (dextran Mᵣ=70,000). Increasing percentage concentration of dextran produces increasing osmotic pressure (Pa) on the gel beads. Over the course of application of increasing osmotic pressure the gel beads were imaged and sized. FIG. 2 shows a plot of the experimental results. The y-axis shows measured bead size as a ratio of the log of the volume of dextran treated gel beads over measured initial volume of gel beads. The x-axis shows the range of osmotic pressure (Pa) tested, as a function of dextran concentration.

The results indicated little variation in compressive elastic modulus (K) for different bead sizes over the osmotic pressure (Pa) range tested. Compressive elastic modulus (K) values for each of the three gel bead sizes are shown in Table 1 below. As presented in Table 1, K measured as kPa, rises somewhat with increasing diameter of gel bead. However, only a small but measurable degree of variation between kPa values was observed in relation to gel bead size.

TABLE 1

| Starting diameter | |
|---|---|
| (microns) | K (kPa) |
| 57.6 | 35.8 |
| 64.8 | 36.1 |
| 72.1 | 40.3 |

### IX. Shear Modulus

Another measure of deformability is shear modulus (G) which is measurable as kPa. It is envisioned that useful particle (e.g., gel bead) deformability can be obtained in a range of G values. For example, from 0.1 kPa to 200 kPa. More specifically, in a range of 1 kPa to 100 kPa. Other useful ranges can include 5 kPa to about 100 kPa, 10 kPa to 100 kPa, 25 kPa to 100 kPa, 25 kPa to 75 kPa and 30kPa to 65 kPa.

### X. Gel Bead Contaminant Removal by Mesh Filtering Gel Beads

Gel beads can clump together when stored in oil (after generation) or when washed in an aqueous buffer. Large debris can also enter gel bead solutions from the surrounding environment. These clumps/debris can clog microfluidic channels within a microfluidic chip which leads to loss of sample. However, gel beads can be filtered to remove clumps of >4 gel beads during their manufacturing using mesh or track etched filters.

Mesh filters were employed to removes gel bead clumps and debris.

Experimental: 30 um gel beads were mesh filtered. The gel beads were passed through nylon woven mesh (90 mm diameter) was tested in 30 um, 20 um, 10 um and 5 um mesh sizes. Gel beads were filtered three times pre and three times post functionalization. Filters were replaced after each round of filtration. Used filter was placed in a 50 mL plastic screw cap tube with 45 mL of DNA ligation buffer. Mesh retained gel beads/debris (e.g., fibers) were examined, in some cases on 300 um FloCam. Negligible bead losses were observed (<5 mL). The process timing was as follows. 20 minutes to rinse and set up filter apparatus. 5 minutes per round of filtration (15 minutes total). 10 minutes to run each retentate sample through FlowCam (optionally).

Gel bead filtration was tested using polycarbonate track etched at 20 um and 10 um.

Results for 30 um gel beads showed that some larger contaminants were removed and the preferred mesh was a nylon mesh filter with 20 um pores (uniform pore size). The control no filtration condition showed a number of clumps found in ˜200,000 gel beads (sub-classified into <5 or ≧6 gel bead clumps). Following three rounds of filtration through a 20 um nylon woven mesh, 90 mm diameter there was a significant reduction in the number of clumps.

Experimental: 54 um gel beads were mesh filtered. The gel beads were passed through nylon mesh in 60 um, 41 um, 30 um and 10 um mesh sizes. Gel bead filtration was tested using polycarbonate track etched at 20 um and 10 um. Results showed that some larger contaminants were removed and the preferred mesh was a nylon mesh filter with 41 um pores (uniform pore size).

The control no filtration condition showed a number of clumps found in ˜100,000 gel beads (sub-classified into <5 or ≧6 gel bead clumps). Following three rounds of filtration through a 41 um nylon woven mesh, 90 mm diameter there was a significant reduction in the number of clumps.

### Y. Prevention of Gel Bead Aggregation

Particle compositions described herein can include attached oligonucleotides, for example barcodes, that are attached by a combinatorial approach. In some combinatorial approaches, ligation protocols may be used to assemble oligonucleotide sequences comprising barcode sequences on beads (e.g., degradable beads as described elsewhere herein). For example, separate populations of beads may be provided to which barcode containing oligonucleotides are to be attached. (see US Patent Application Publication No. US20140378349, incorporated herein by reference) Following a ligation protocol gel beads have been found to have a tendency for clumping and being gummy. Without being held to a particular theory, one possible explanation is that the inclusion of fluorophores in the ligation protocol causes clumping of the gel beads. Another possibility is that the presence of reducing agents in the ligation protocol adversely affect gel beads, resulting in the observed clumping and gumminess. In the ligation reaction, DTT was present up to 40 μM.

Experimental: Changes in gel bead size and clumping were studied upon exposure to reducing agent. Gel beads were incubated in about 20 μM TCEP (tris(2-carboxyethyl)phosphine) reducing agent for 30 minutes, resulting in clumping of the gel beads. Gel beads incubated at lower concentrations of TCEP were found to undergo size increase.

In a separate study the effect of gummy gel beads in preparation of gel bead injection into an emulsion was tested. It was found that gummy gel beads from a ligation protocol caused clogging and uneven injection of the gel beads into an emulsion. It was also found that running the ligation protocol under 40 μM DTT conditions caused uneven injection and clumping of the gel beads on a fluidic chip.

It was discovered that clumping and gumminess of gel beads observed could be remedied by altering the ligation buffer of the ligation protocol. By removing the reducing agent, creating a ligation protocol that was reducing agent free or substantially reducing agent free, the gel beads were free of clumping and gumminess (data not shown). This was measurable, for example by measuring bead injection rate into emulsions (data not shown). It was also discovered that the ligation enzyme component of the ligation protocol include a level of reducing agent that could be eliminated from the enzyme prior to conducting the ligation protocol to achieve the lack of clumping and gumminess. In sum, the results showed that gel beads could be better stabilized, and show reduced clumping and gumminess when the ligation protocol was performed without any reducing agents. Surprisingly, it was discovered that removal of reducing agent from the ligase enzyme not only improved gel bead characteristics but had no adverse effect on the activity of the enzyme in the ligation protocol with gel beads.

### Z. pH Optimization for Preserving Oligonucleotide-Gel Bead Linkage

In preparing and storing labeled gel beads, for example, oligonucleotide tagged or barcoded gel beads, a contamination effect has been observed where oligonucleotides undesirably release from the gel beads during storage without any stimulus for release. pH optimization was studied as a solution to this issue.

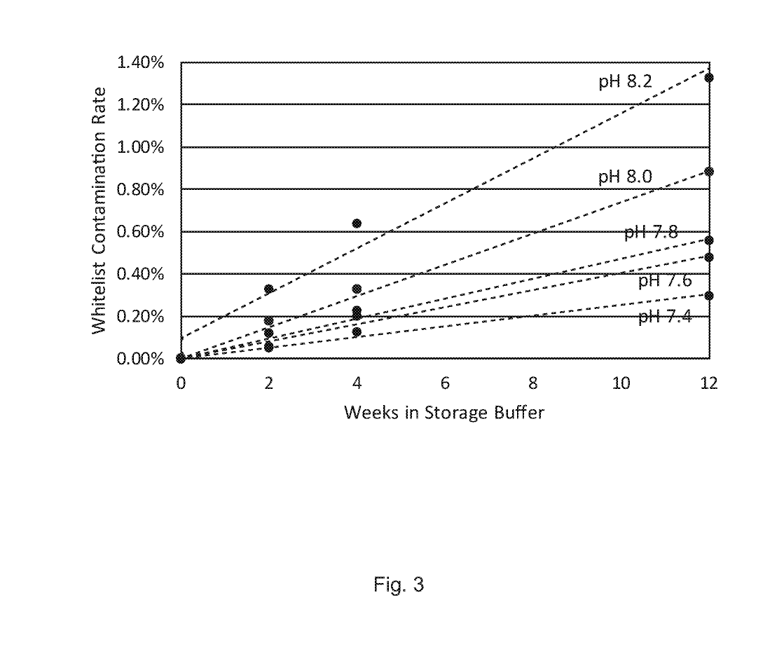

Experimental: Over a course of week, up to 12 weeks, barcoded gel beads were stored in a storage buffer at various pH conditions and the contamination rate (released oligonucleotides) was measured per week. The results, illustrated in FIG. 3, showed that pH 7.4 was optimal for reducing contamination over the 12 weeks storage period. As little as 0.025% contamination was detected when stored at pH 7.4. As pH was increased, the contamination rate increased. The most contamination occurred in the highest pH tested, pH 8.2, where as much as 0.110% contamination was detected. The contamination results are summarized in Table 2.

TABLE 2

| | Contamination Rate |
|---|---|
| pH | (per week) |
| 8.2 | 0.110% |
| 8.0 | 0.072% |
| 7.8 | 0.050% |
| 7.6 | 0.041% |
| 7.4 | 0.025% |

### AA. Using Gel Beads as Filters

Gel beads can include a range of mesh sizes in their physical structure. The mesh size provided in a gel bead can be useful as a flow restrictor whereby objects of a small enough size, can diffuse or flow into and even through the gel bead, while objects of larger sizes would not be capable of diffusing or flowing into the gel bead. Experimental rationale was that larger mesh size of gel beads should result in diffusion of FITC-Dextran into the gel beads, while with smaller mesh size gel beads should not permit diffusion into the gel beads.

Experimental: FITC-Dextran was used to test with two different mesh sizes of gel beads. 0.1% w/w stock solutions of FITC-Dextrans as shown in Table 3 were used in the study.

TABLE 3
FITC-Dextran

| | | | | | |
|---|---|---|---|---|---|
| Average Molecular Weight | g mol−1 | 40,000 | 20,000 | 10,000 | 4,000 |
| Lot # | | SLBH1157V | SLBH1156V | SLBD1132V | BCBM9769V |
| Mass | g | 0.1 | 0.1 | 0.1 | 0.1 |
| Mass of water | g | 9.919 | 9.9263 | 9.9455 | 9.958 |
| Concentration | % w/w | 0.9981 | 0.9974 | 0.9955 | 0.9942 |

The stock solutions were diluted down to a final concentration of 0.055% w/w. Stoke's radius was measured for each molecular weight FITC-Dextran using dynamic light scattering. (data not shown) 10 uL of packed gel beads was added to 90 uL of FITC-Dextran and incubated in the dark overnight. Bright field (phase-contrast) and fluorescence (488 nm) images of gel beads were taken. For six gel bead lots tested it was determined that the gel bead mesh size was consistent across all gel bead lots and sized at <4.4 nm. (data not shown).

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. For example, particle delivery can be practiced with array well sizing methods as described. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition, comprising a suspension of deformable particles, the suspension being characterized by one or more of:
(i) a dispersity of particles where at least 95% of the deformable particles in the suspension have a particle size that is within 10% of a mean particle size for the suspension;
(ii) a plurality of deformable particles having an elastic modulus of between about 5 kPa and 100 kPa;
(iii) a solution viscosity of between about 0.1 cP and about 100 cP; and
(iv) deformable particles having a pore size of from about 1 nm to about 20 nm.

2. The composition of claim 1, wherein the deformable particles are passable through a microfluidic physical feature narrower than the deformable particles.

3. The composition of claim 1, wherein the microfluidic physical feature is: a filter, an obstacle, a passage, a channel, a space or any combinations thereof.

4. The composition of claim 1, wherein the deformable particle is a bead.

5. The composition of claim 4, wherein the bead is a gel bead.

6. The composition of claim 1, wherein the deformable particle has a diameter selected from the group consisting of about 1 μm to about 1 mm, about 10 μm to about 100 μm, about 20 μm to about 100 μm, about 30 μm to about 80 μm and about 40 μm to about 60 μm in diameter.

7. The composition of claim 6, wherein the deformable particle has a diameter of about 10 μm to about 100 μm.

8. The composition of claim 7, wherein the deformable particle has a diameter of about 30 μm to about 100 μm.

9. The method of claim 1, wherein the suspension is characterized by a shear modulus between about 5 kPa to about 100 kPa.

10. A method of removing contaminants from the suspension of deformable particles of claim 1, comprising:
i) passing the deformable particles through a mesh filter having a pore size smaller than the diameter of the deformable particles; and
ii) collecting the deformable particles.

11. The method of claim 10, wherein the mesh filter comprises a pore size of about 10 μm to about 50 μm.

12. The method of claim 10, wherein the deformable particle has a diameter of about 10 μm to about 100 μm.

13. The method of claim 10, wherein the deformable particle has a diameter of about 30 μm to about 100 μm.

14. The method of claim 10, wherein the pore size is about 30 μm.

15. The method of claim 10, wherein the pore size is about 41 μm.

16. The method of claim 10, wherein the deformable particles are gel beads.

17. A method of storing an oligonucleotide labeled deformable gel bead composition comprising:
i) providing a composition of deformable gel beads linked to an oligonucleotide; and
ii) storing the composition at about pH 7.4 for at least 12 weeks, wherein release of linked oligonucleotides is at most 0.025%.

18. The method of claim 17, wherein the deformable gel beads have a diameter of about 10 μm to about 100 μm.

19. The method of claim 17, wherein the deformable gel beads have a diameter of about 30 μm to about 100 μm.

20. A composition comprising a suspension of deformable particles characterized by:
i) the suspension having a solution comprising a ligation buffer, a ligase enzyme, oligonucleotides and an absence of any reducing agent, wherein the solution supports ligation of oligonucleotides to the deformable particles even in the absence of the reducing agent;
ii) the deformable particles having an elastic modulus of between about 5 kPa and 100 kPa; and
iii) the deformable particles being resistant to aggregation, wherein the deformable particles would otherwise be prone to aggregation in the presence of a reducing agent.

21. The composition of claim 20, the suspension further being characterized by one or more of:
i) a dispersity of particles where at least 95% of the particles in the suspension have a particle size that is within 10% of a mean particle size for the suspension;
ii) a solution viscosity of between about 0.1 cP and about 100 cP; and
iii) particles having a pore size of from about 1 nm to about 20 nm.

22. A method of filtering using the suspension of deformable particles of claim 1 comprising:
i) using the suspension of deformable particles as a flow restrictor; and
ii) passing a solution to be filtered through the suspension.

23. The method of claim 22, wherein the deformable particles have a pore size of from about 2 nm to about 6 nm.

24. The method of claim 22, wherein the deformable particles have a pore size of from about 5 nm.

25. The method of claim 22, wherein the deformable particles provide a size cut off of less than 4.4 nm.

26. A composition, comprising a suspension of particles, the suspension being characterized by having one or more of:
(i) a dispersity of particles where at least 95% of the particles in the suspension have a particle size that is within 10% of a mean particle size for the suspension;
(ii) a plurality of particles having an elastic modulus of between about 5 kPa and 100 kPa;
(iii) a solution viscosity of between about 0.1 cP and about 100 cP; and
(iv) particles having a pore size of from about 1 nm to about 20 nm.
